(19) United States
(12) Patent Application Publication
(10) Pub. No.: US 2024/0307710 A1
(43) Pub. Date: Sep. 19, 2024

(54) STIMULATOR AND METHOD FOR APPLYING ACOUSTIC ENERGY IN A TARGET REGION ON AN INDIVIDUAL

(71) Applicant: MEDERGIE LIMITED, LOUTH (IE)

(72) Inventor: Daniel HUET, TOULON (FR)

(73) Assignee: MEDERGIE LIMITED, LOUTH (IE)

(21) Appl. No.: 18/263,421

(22) PCT Filed: Jan. 27, 2022
(86) PCT No.: PCT/EP2022/051920

§ 371 Date: Jul. 28, 2023

(30) Foreign Application Priority Data
Jan. 28, 2021 (FR) 2100786

(51) Int. Cl. A61N 7/02; A61N 7/00

(52) U.S. Cl. CPC A61N 7/02; A61N 2007/0026; A61N 2007/0073; A61N 2007/0078; A61N 2007/027

(57) ABSTRACT

A stimulator for applying acoustic energy, in which: a first emitter of acoustic energy is equipped with a first array of electroacoustic transducers and a second emitter of acoustic energy is equipped with a second array of electroacoustic transducers, the emitters being arranged symmetrically in a mirror arrangement; an electronic management unit activates the transducers of the first array so that the transducers issue a first beam of acoustic waves in the direction of a target region on the individual, and the transducers of the second array so that the transducers issue a second beam of acoustic waves in the direction of the target region; the activation of the transducers is sequenced so that the transducers are activated in the form of successive concentric rings or circles of different diameters or in the form of a spiral, or in the form of a rotating helix.

### TECHNICAL FIELD

The aim of the invention is a stimulator, a non-therapeutic method and a therapeutic treatment device, intended to apply acoustic energy in a target zone of an individual (human or animal).

The invention relates to the general technical field of stimulating the body by applying acoustic energy.

### BACKGROUND

Therapies using acoustic energy are known. Notably, ultrasound probes are known for acting on the tissues of the body by virtue of thermal effects. The heat produced by high frequencies allows, for example, pain to be reduced, blood circulation to be promoted, articular tissues to be softened, or even certain cancerous tumors to be destroyed. Such probes are described, for example, in patent documents U.S. Pat. No. 5,285,772 or EP 1847294. Patent document EP 225794 describes another probe comprising ultrasound transducers. Patent documents US 2012/0289869 and US 2020/0384292 also disclose devices comprising ultrasound transducers used to act on brain structures of subjects. The article by RAMAEKERS et al., entitled, “Evaluation of a novel therapeutic focused ultrasound transducer based on Fermat's spiral”, Physics in Medicine & Biology, Phys. Med. Biol. 62 (2017) 5021-5045, discloses yet another ultrasound transducer device for acting on a subject. Even though they are effective, these devices are nevertheless complex and expensive and generally have a field of use limited to a specific therapeutic application.

Non-therapeutic relaxation methods are also known in which sounds perceptible by the human ear allow access to a certain kind of physical and mental well-being. Such a device is described, for example, on the following web page: https://www.nowbysolu.com. These devices also have a limited field of use and have relative effectiveness.

The invention aims to overcome all or some of the aforementioned disadvantages. The invention notably aims to achieve one or more of the following objectives:
providing a stimulator with enhanced effectiveness compared to the stimulators of the prior art, the design of which stimulator is simple and inexpensive;
designing a stimulator that can be used both within the context of therapeutic treatments and non-therapeutic treatments;
proposing a stimulator that is effective over a significant range of individuals.

### SUMMARY

The solution proposed by the invention is a stimulator intended to apply acoustic energy in a target zone of an individual, wherein:
a first acoustic energy emitter is equipped with a first array of electroacoustic transducers and a second acoustic energy emitter is equipped with a second array of electroacoustic transducers, said emitters being symmetrically arranged in a mirror arrangement, with the first array facing the second array;
an electronic management unit is configured to activate the transducers of the first array so that said transducers diffuse a first acoustic wave beam toward a target zone of the individual, and to activate the transducers of the second array so that said transducers diffuse a second acoustic wave beam toward said target zone;
the activation of the transducers of the first array and of the second array is sequenced so that said transducers of each emitter are activated in the form of successive concentric circles or rings with different diameters or in the form of a spiral or in the form of a helix rotating about an acoustic axis of the two emitters.

The use of two emitters arranged as a mirror allows the acoustic energy of two acoustic wave beams to be combined and concentrated in the target zone. Moreover, due to the mirror arrangement, these acoustic wave beams act in two opposite directions, so that the target zone can be stimulated from two distinct faces, notably from the front and from the rear. As a result, the effectiveness of the stimulator is enhanced compared to the aforementioned stimulators of the prior art. Furthermore, the inventor has found that the specific diffusion of the acoustic waves in the form of concentric circles, disks or rings, of a spiral or of a rotating helix allowed the effect (therapeutic or non-therapeutic) of the acoustic energy applied in the target zone to be enhanced. When these two features (mirror arrangement and specific diffusion of the acoustic waves) act together, a synergistic effect is acquired in terms of stimulation of the target zone.

Further advantageous features of the method according to the invention are listed hereafter. Each of these features can be considered alone or in combination with the noteworthy features defined above. Each of these features contributes, if applicable, to the resolution of specific technical problems as defined above in the description and to which the other features as defined above do not necessarily participate. The latter can, if applicable, form one or more divisional patent applications:
According to one embodiment, the acoustic waves are sound waves.
According to one embodiment, the first emitter and the second emitter are installed on the walls of a cabin.
According to one embodiment, a seat is placed in the cabin so that, when the individual is seated on said seat, the first emitter is located opposite said individual and the second emitter is located at the back of said individual.
According to another embodiment, the acoustic waves are ultrasound waves.
According to one embodiment, the ultrasound frequency of the waves of the first beam is different from the ultrasound frequency of the waves of the second beam.
According to one embodiment, the acoustic waves emitted by the transducers of the first array have the same features as the acoustic waves emitted by the transducers of the second array.
According to one embodiment, the transducers of the first array and the transducers of the second array are directive diffusion electroacoustic transducers.
According to one embodiment: —the first beam converges toward a first focal point so as to receive a stimulation in a first focal zone; —the second beam converges toward a second focal point, which is distinct from said first focal point, so as to receive a stimulation in a second focal zone; —a third stimulation focal zone corresponds to the overlapping zone where the first beam and the second beam intersect.
According to one embodiment, the target zone is located in the acoustic axis of the two emitters and midway from said emitters.
According to one embodiment, the electronic management unit is configured to activate the transducers of the first array and the transducers of the second array.
According to another embodiment, the electronic management unit is configured to activate the transducers of the first array in accordance with a first sequence and the transducers of the second array in accordance with a second sequence different from said first sequence.
According to one embodiment, the electronic management unit is configured to activate the transducers of the first array and the transducers of the second array with a time lag.

Another aspect of the invention relates to a non-therapeutic method for applying acoustic energy in a target zone of an individual, the method comprising the following steps of:
equipping a first acoustic energy emitter with a first array of electroacoustic transducers and a second acoustic energy emitter with a second array of electroacoustic transducers;
symmetrically positioning the first emitter and the second emitter in a mirror arrangement, with the first array facing the second array;
activating the transducers of the first array so that said transducers diffuse a first acoustic wave beam toward a target zone of the individual, and the transducers of the second array so that said transducers diffuse a second acoustic wave beam toward said target zone, which acoustic waves are sound waves;
sequencing the activation of the transducers of the first array and of the second array so that said transducers of each emitter are activated in the form of successive concentric circles, disks or rings with different diameters or in the form of a spiral or in the form of a helix rotating about an acoustic axis of the two emitters.

Yet another aspect of the invention relates to a device for the therapeutic treatment of a neurological disease comprising a brain cell stimulator, with said stimulator being a stimulator according to any of the preceding features.

### BRIEF DESCRIPTION OF THE FIGURES

Further advantages and features of the invention will become more clearly apparent upon reading the following description of a preferred embodiment, with reference to the appended drawings, which are provided by way of indicative and non-limiting examples and in which:

FIG. 1a schematically shows a first arrangement of electroacoustic transducers.

FIG. 1b schematically shows a second arrangement of electroacoustic transducers.

FIG. 1c schematically shows a third arrangement of electroacoustic transducers.

FIG. 2 schematically shows an example of the mounting of electroacoustic transducers.

FIG. 3a schematically shows a first shape of an acoustic wave beam.

FIG. 3b schematically shows a second shape of an acoustic wave beam.

FIG. 3c schematically shows a third shape of an acoustic wave beam.

FIG. 4 schematically shows a device according to the invention according to a first embodiment.

FIG. 5 schematically shows a device according to the invention according to a second embodiment.

FIG. 6a illustrates a first example of the interaction of the acoustic waves emitted by two emitters.

FIG. 6b illustrates a second example of the interaction of the acoustic waves emitted by two emitters.

FIG. 6c illustrates a third example of the interaction of the acoustic waves emitted by two emitters.

### DETAILED DESCRIPTION OF THE EMBODIMENTS

The invention can implement one or more computer applications executed by computer equipment. For the sake of clarity, it is to be understood, within the meaning of the invention, that “an item of equipment performs a task” means “the computer application executed by a processing unit of the item of equipment performs a task”. Likewise, “the computer application performs a task” means “the computer application executed by the processing unit of the item of equipment performs a task”.

Still for the sake of clarity, the present invention is able to refer to one or more “computer processes”. These computer processes correspond to the actions or results acquired by executing the instructions of one or more computer applications. Furthermore, it is also to be understood, within the meaning of the invention, that “a computer process is adapted to perform a task” means “the instructions of a computer application executed by a processing unit perform a task”.

If applicable, and to optionally complete their current definition, the following further information is added to some of the terms used in the description and the claims:
“Computer resource” in a non-limiting manner can be understood to be: component, hardware, software, file, connection to a computer network, amount of RAM, hard disk space, bandwidth, processor speed, number of CPUs, etc.;
“Electronic management unit” in a non-limiting manner can be understood to be: processor, microprocessors, CPU (Central Processing Unit);
“Computer application” can be understood to be: software, computer program product, computer program or software, the instructions of which are notably executed by a processing unit;
As used herein, unless otherwise indicated, the use of the ordinal adjectives “first”, “second”, etc., to describe an object simply indicates that different occurrences of similar objects are mentioned and does not imply that the objects thus described must be in a given sequence, whether by time, in space, in a classification or in any other way;
“X and/or Y” means: X alone or Y alone or X+Y;
In general, it is acknowledged that, in the various appended drawings, the objects are arbitrarily drawn in order to facilitate the reading thereof.

An aim of the invention is a stimulator intended to apply acoustic energy to the body of an individual. This stimulator can be used for therapeutic purposes and for non-therapeutic purposes, as explained above.

The stimulator comprises two acoustic energy emitters symmetrically arranged in a mirror arrangement. A first emitter is equipped with a first array of electroacoustic transducers and a second emitter is equipped with a second array of electroacoustic transducers. The spacing between the two emitters can be fixed or variable.

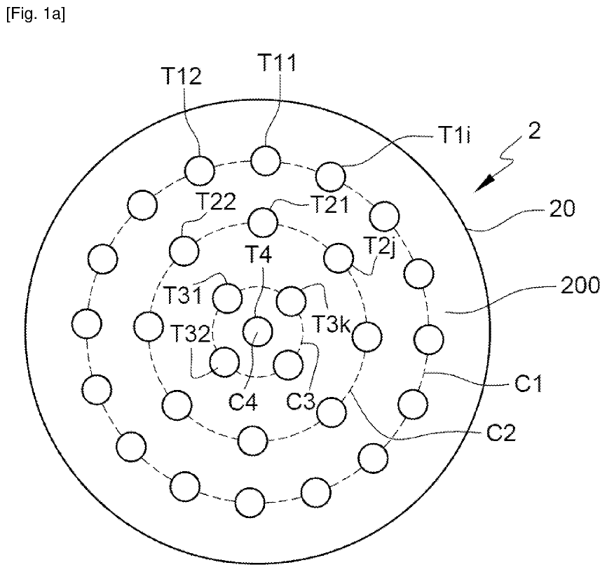

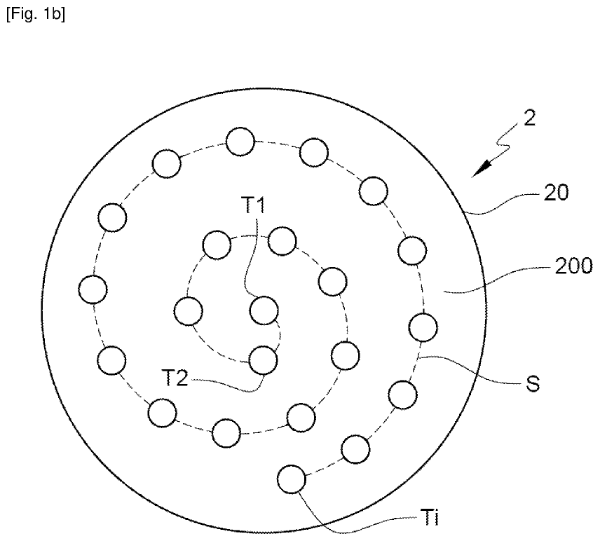

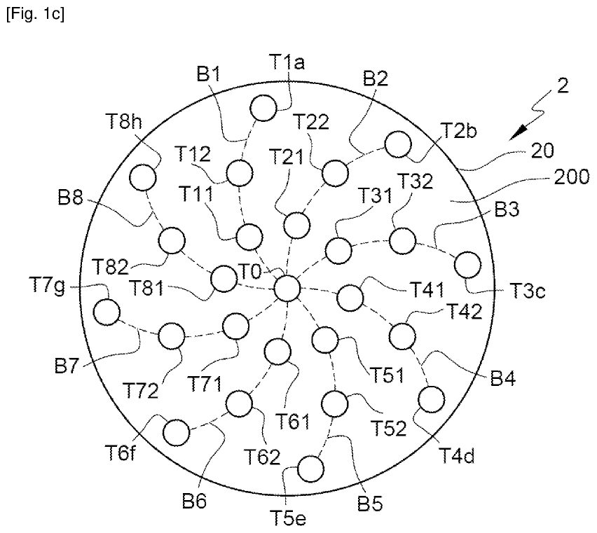

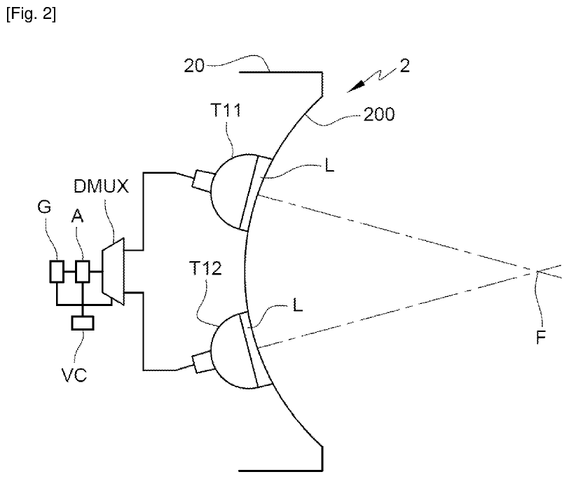

In FIGS. 1a to 1c and 2, the emitter 2 comprises a support body 20 having a front surface 200, on which the transducers T are arranged. The front surface 200 can be flat, concave, parabolic, etc. In FIG. 2, it is concave or parabolic, which is a simple constructive solution allowing the acoustic wave emitted by the transducers T11, T12 to be focused toward a focal point F.

The front surface 200 is preferably circular, but can assume another shape, for example, it can be oval, square, rectangular, with lobes, etc. The size of the front surface depends on the use made of the stimulator, as explained above.

The transducers convert an electrical signal into an acoustic wave. When a transducer is activated, it emits an acoustic wave. This emission can be referred to as “wave shot” throughout the remainder of the description. In FIG. 2, and conventionally, the transducers T11, T12 are connected via an amplifier stage A to a signal generator G. These elements are controlled by an electronic management unit UC. The transducers T11, T12 are activated independently of one another, according to predefined sequences, as explained above. According to one embodiment, a demultiplexer DMUX is provided, an input of which is connected to the amplifier A and the outputs of which are connected to the transducers T11, T12. The unit UC allows the one or more transducers T11, T12 to be activated to be selected by selecting the one or more corresponding outputs of the demultiplexer DMUX.

According to one embodiment, the unit UC contains, for example, in the form of files stored in a memory zone, all the information suitable for individually controlling each of the individual transducers, according to given parameters (sequence, rate, frequencies, etc.). These parameters are preferably modifiable.

### Transducers: First Embodiment

According to a first embodiment, the emitter 2 is an acoustic enclosure and the transducers T11, T12 are loudspeakers that each diffuse a sound wave, i.e., a sound audible to the human ear (frequencies ranging between approximately 16 Hz and 20,000 Hz, at an acoustic level preferably ranging between 1 dB and 80 dB).

According to one embodiment, the loudspeakers T11, T12 are directive diffusion loudspeakers so as to focus their sound wave toward a focal point F. According to one embodiment, the focusing is mechanical focusing carried out by acoustic lenses. According to another embodiment, the focusing is electronic focusing, for example, adapted to assign phase delays between the loudspeakers. According to another embodiment, the focusing is mixed focusing (mechanical and electronic).

According to one embodiment, the directivity of the sound waves can be acquired by using loudspeakers integrating HSS® (HyperSonic Sound) technology. The principle of HSS® technology involves modulating the sound wave to be diffused around ultrasound carrier frequencies. The sound wave becomes audible when it encounters an obstacle, notably an individual. Therefore, the sound can be heard directly when the ear of the individual crosses the ultrasound beam. Other directive diffusion acoustic enclosures are described, for example, in patent documents FR 3087608 or U.S. Pat. No. 10,129,657, to which a person skilled in the art will be able to refer, if applicable.

### Transducers: Second Embodiment

According to a second embodiment, the transducers T11, T12 are therapeutic transducers, notably ultrasound transducers, typically piezoelectric transducers, which operate at a frequency ranging from approximately 0.1 MHz to approximately 50 MHz. High Intensity Focused Ultrasound (HIFU) transducers operating at frequencies ranging between 0.1 MHz and approximately 10 MHz are preferably used. This more specifically involves therapy transducers used, for example, to treat an undesirable human or animal tissue, such as diseased tissue or adipose tissue. The emitter 2 then can be in the form of an ultrasound therapeutic probe.

According to one embodiment, the ultrasound transducers T11, T12 are directive diffusion transducers. The focusing of their ultrasound wave toward a focal point F notably can be mechanical focusing carried out by a convergent acoustic lens L (FIG. 2) or a Fresnel lens. The focusing also can be electronic (for example, for assigning phase delays) or mixed (mechanical and electronic).

### First Arrangement of Transducers—FIG. 1 a.

The transducers T are arranged in the form of n concentric circles Cn. In FIG. 1a, four concentric circles are schematically shown (n=4), but a lower or higher number of circles can be provided. For example, 2≤n≤100. The transducers can be attached or spaced apart from one another, on the same circle and/or from one circle to the next. The diameter of the circles Cn ranges, for example, between 2 cm and 2 m and depends on the type of emitter used and/or on how it is applied.

In FIG. 1a, the first circle C1 comprises i transducers (T11, T12, . . . , T1i), the second circle C2 comprises j transducers (T21, T22, . . . , T2j), the third circle C3 comprises k transducers (T31, T32, . . . , T3k), and the fourth circle C4 comprises a single transducer T4, which corresponds to the center of the array. A greater number of transducers nevertheless can be provided at the center of the array. According to another example, no transducer is positioned at this location. The numbers i, j and k are integers, for example, ranging between 2 and 1,000, preferably with i>j>k. However, it is possible to have the same number of transducers on each circle, in which case i=j=k. The number of transducers on each circle depends on the type of emitter used and/or on how it is applied. The transducers can be identical from one circle to the next or can be different (for example, of increasing or decreasing size). Similarly, on the same circle, the transducers can be identical or different.

In this arrangement, the transducers are activated sequentially, in the form of successive concentric of circles, disks or rings with different diameters.

The tables below illustrate various examples of sequencing at instants to, t1, t2, t3 (with t0<t1<t2<t3). These instants represent the rate of the wave shots. The numbers “0” and “1” indicate the state of the transducers of a circle Cn (0=deactivated transducers and 1=activated transducers). For example, at t0, C1=0, indicates that all the transducers of the circle C1 are deactivated at the instant t0, and at t3, C1=1, indicates that all the transducers of the circle C1 are activated at the instant t1.

It should be noted that the tables below are only used to illustrate sequences allowing successive concentric circles or rings with different diameters to be acquired, with the circles C1-C4 being activated in an ascending manner (from the smallest to the largest diameter). These tables by no means limit the invention, other sequences can be contemplated by a person skilled in the art. Notably, sequences in which the circles are activated in a descending manner (from the largest to the smallest diameter).

TABLE 1

| | t0 | t1 | t2 | t3 |
|---|---|---|---|---|
| C1 | 0 | 0 | 0 | 1 |
| C2 | 0 | 0 | 1 | 0 |
| C3 | 0 | 1 | 0 | 0 |
| C4 | 1 | 0 | 0 | 0 |

TABLE 2

| | t0 | t1 | t2 | t3 |
|---|---|---|---|---|
| C1 | 0 | 0 | 0 | 1 |
| C2 | 0 | 0 | 1 | 1 |
| C3 | 0 | 1 | 1 | 1 |
| C4 | 1 | 1 | 1 | 1 |

TABLE 3

| | t0 | t1 | t2 | t3 |
|---|---|---|---|---|
| C1 | 0 | 0 | 1 | 1 |
| C2 | 0 | 1 | 1 | 0 |
| C3 | 0 | 1 | 0 | 0 |
| C4 | 1 | 0 | 0 | 0 |

TABLE 4

| | t0 | t1 | t2 | t3 |
|---|---|---|---|---|
| C1 | 0 | 1 | 0 | 0 |
| C2 | 0 | 0 | 0 | 1 |
| C3 | 0 | 0 | 1 | 0 |
| C4 | 1 | 0 | 0 | 0 |

These sequences can be repeated several times, and/or as many times as necessary. The instants t0, t1, t2, t3 can last from 0.1 milliseconds to 5 seconds each, depending on the use made of the stimulator. For example, when the transducers are loudspeakers, the instants t0, t1, t2, t3 can be of the order of one second. Also, if the transducers are ultrasound transducers, the instants t0, t1, t2, t3 can be of the order of one millisecond. The instants t0, t1, t2, t3 can have the same duration or different durations. The instants t0, t1, t2, t3 are in succession so that there are always active transducers.

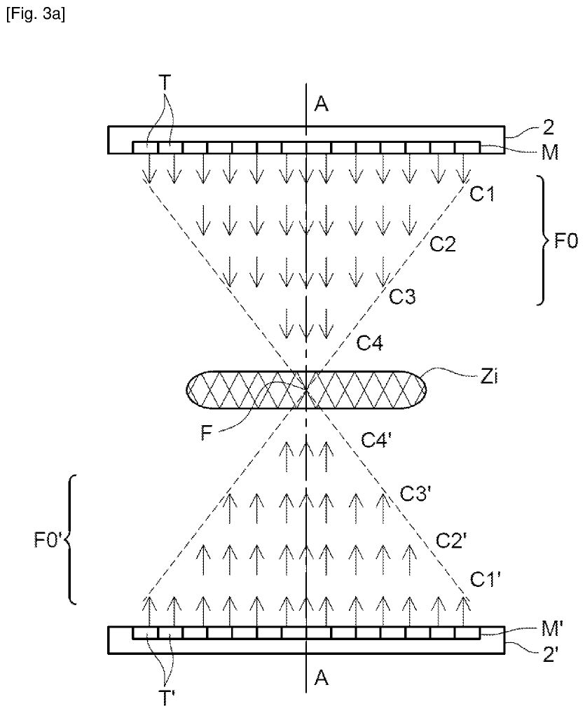

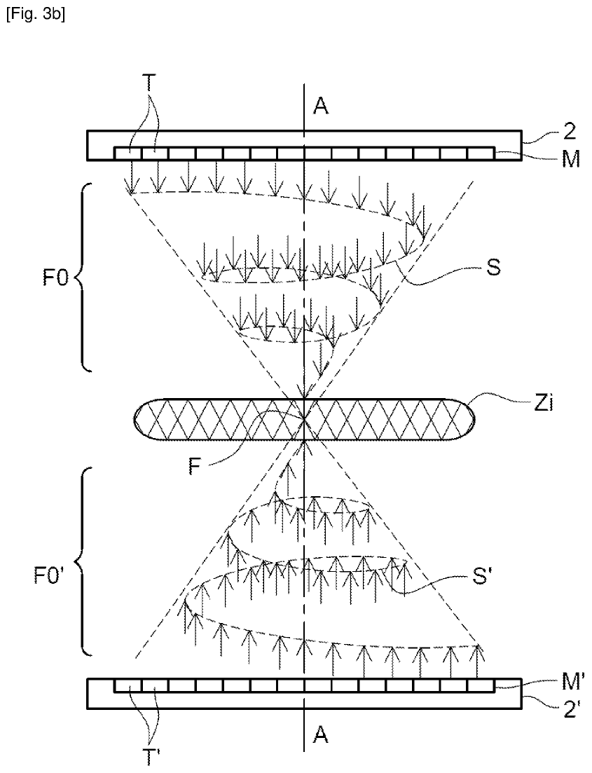

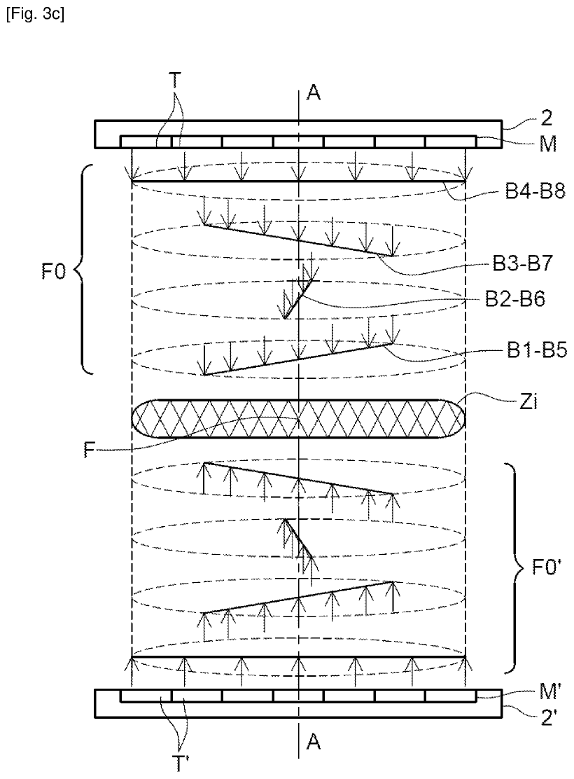

FIG. 3a illustrates an acoustic wave beam shape acquired with an array M of transducers according to the first arrangement. The first wave shot is that of the transducers of the smaller diameter circle C4, the second shot is that of the transducers of the circles C3+C4, the third shot is that of the transducers of the circles C2+C3+C4 and the fourth shot is that of the transducers of the circles C1+C2+C3+C4, and so on. This succession of shots corresponds to [Table 2]. The beam is thus made up of a series of waves in the form of concentric disks that converge toward a focal point F located on the acoustic axis A-A of the emitter 2.

The focal point F is located in the target zone Zi. This target zone Zi can be a one-dimensional, two-dimensional or three-dimensional zone. It corresponds to a part of the body of the individual (for example, to the head) and can be located inside the body of the individual (for example, a diseased tissue to be treated).

The entire surface of the target zone Zi is reached by the beam, but in a gradual manner (gradually from the center or progressively toward the center). In the example of FIG. 3a, the target zone Zi will first receive the acoustic energy from the first disk (C4) on a first sector, then from the second disk (C3+C4) on a second sector, then from the third disk (C2+C3+C4) on a third sector, then from the fourth disk (C1+C2+C3+C4) on a fourth sector, which sectors are concentric, and so on. The inventor has found that reaching the target zone Zi in this localized and progressive manner allowed fast and deep penetration of the acoustic waves into the body of the individual and allowed fast and homogeneous stimulation of said target zone, in the sense that the acoustic energy is better distributed in said zone and is not highly focused in a single focal point. Also, in the case whereby the transducers are ultrasound transducers, the formation of very hot spots at the focal point F is avoided. The acoustic energy of said waves thus acts with enhanced effectiveness in the target zone Zi, which is better stimulated, notably compared to the aforementioned stimulators of the prior art.

### Second Arrangement of Transducers—FIG. 1b.

The transducers T are arranged in the form of a spiral S comprising n turns. The central point is advantageously located at the center of the array. In FIG. 1b, two turns are schematically shown (n=2), but a lower or higher number of turns can be provided. For example, 2≤n≤100. The transducers can be attached or spaced apart from one another along the spiral. The maximum diameter of the spiral ranges, for example, between 2 cm and 2 m and depends on the type of emitter used and/or on how it is applied.

In FIG. 1b, a number i of transducers is arranged along the spiral. The number i is an integer ranging between 4 and 1,000 and which depends on the type of emitter used and/or on how it is applied. The transducers can be identical or different along the spiral (for example, of increasing or decreasing size).

In this arrangement, the transducers are activated sequentially, in the form of a spiral. The tables below illustrate various examples of sequencing at instants t0, t1, t2, . . . , ti (with t0<t1<t2< . . . <ti). These instants represent the rate of the wave shots. The numbers “0” and “1” indicate the state of the transducers (0=deactivated transducers and 1=activated transducers). For example, at t0, T1=1, indicates that the transducer T1 is activated at the instant t0, and at t3, T1=0, indicates that the transducer T1 is deactivated at the instant to.

It should be noted that the tables below are only used to illustrate sequences allowing a spiral to be acquired. These tables by no means limit the invention, other sequences can be contemplated by a person skilled in the art. Notably, sequences in which the spiral is activated in a descending manner (from the transducer Ti farthest from the center, toward said center).

TABLE 5

| | t0 | t1 | t2 | . . . | ti |
|---|---|---|---|---|---|
| T1 | 1 | 0 | 0 | 0 | 0 |
| T2 | 0 | 1 | 0 | 0 | 0 |
| T3 | 0 | 0 | 1 | 0 | 0 |
| . . . | 0 | 0 | 0 | 1 | 0 |
| Ti | 0 | 0 | 0 | 0 | 1 |

TABLE 6

| | t0 | t1 | t2 | . . . | ti |
|---|---|---|---|---|---|
| T1 | 1 | 1 | 1 | 1 | 1 |
| T2 | 0 | 1 | 1 | 1 | 1 |
| T3 | 0 | 0 | 1 | 1 | 1 |
| . . . | 0 | 0 | 0 | 1 | 1 |
| Ti | 0 | 0 | 0 | 0 | 1 |

These sequences can be repeated several times, and/or as many times as necessary. The instants t0, t1, t2, . . . , ti can last from 0.1 milliseconds to 5 seconds, depending on the use made of the stimulator. For example, when the transducers are loudspeakers, the instants t0, t1, t2, . . . , ti can be of the order of one second. Also, if the transducers are ultrasound transducers, the instants t0, t1, t2, . . . , t3 can be of the order of one millisecond. The instants t0, t1, t2, . . . , t3 can have the same duration or different durations. The instants t0, t1, t2, . . . , t3 are in succession so that there are always active transducers.

Other arrangements of arrays allow the transducers to be activated in the form of a spiral. For example, in the case of FIG. 1a, the transducer T4 is activated first, then the transducers T31 to T3k of the third circle C3 are successively activated, then the transducers T21 to T2j of the second circle C2 are successively activated, then the transducers T11 to T1i of the first circle C1 are successively activated.

FIG. 3b illustrates an acoustic wave beam shape acquired with an array M of transducers according to the second arrangement. The first wave shot is that of the transducer T1 located at the center of the spiral S, then the other transducers are successively activated along said spiral. This succession of shots corresponds to [Table 5]. The beam is thus made up of a series of waves in the form of a spiral that converges toward the target zone Zi. The beam assumes a conical spiral or vortex shape.

In this case again, the entire surface of the target zone Zi is reached by the beam, but in a progressive manner. The target zone Zi will first receive the acoustic energy from the first transducer (T1), then successively from the other transducers, with each shot reaching a localized region of said zone. The inventor has found that reaching the target zone Zi in this localized and progressive manner allowed fast and deep penetration of the acoustic waves into the body of the individual and allowed fast and homogeneous stimulation of said target zone, in the sense that the acoustic energy is better distributed in said zone and is not highly focused in a single focal point. Also, in the case whereby the transducers are ultrasound transducers, the formation of very hot spots at the focal point F is avoided. The acoustic energy of said waves thus acts with enhanced effectiveness in the target zone Zi, which is better stimulated, notably compared to the aforementioned stimulators of the prior art.

### Third Arrangement of Transducers—FIG. 1c.

The transducers T are arranged in the form of a star with n branches. The central point of this star shape is advantageously located at the center of the array. In FIG. 1c, eight branches are schematically shown (n=8), but a lower or higher number of branches can be provided. For example, 4≤n≤100. The branches B1-B8 can be curved, straight, C-shaped, V-shaped, broken lines, etc. The transducers can be attached or spaced apart from one another, on the same branch and/or from one branch to the next. The length of the branches B1-B8 ranges, for example, between 2 cm and 2 m and depends on the type of emitter used and/or how it is applied.

In FIG. 1c, the first branch B1 comprises a transducers (T11, T12, . . . , T1a), the second branch B2 comprises b transducers (T21, T22, . . . , T2b), the eighth branch B8 comprises h transducers (T81, T82, . . . , T8h). The center of the array comprises a single transducer TO. However, a greater number of transducers can be provided at the center of the array. According to another example, no transducer is positioned at this location. The numbers a, b, c, . . . , h are integers, for example, ranging between 2 and 1,000. It is possible to have the same number of transducers on each branch (in which case a=b=b= . . . =h) or to have a different number. The transducers can be identical from one branch to the next or can be different (for example, of increasing or decreasing size). Similarly, on the same branch, the transducers can be identical or different (for example, of increasing or decreasing size).

In this arrangement, the transducers are activated sequentially, in the form of a rotating helix. This helix rotates about the acoustic axis A-A. The tables below illustrate various examples of sequencing at instants t0, t1, t2, . . . , t7 (with t0<t1<t2<t3). These instants represent the rate of the wave shots. The numbers “0” and “1” indicate the state of the transducers of a circle Cn (0=deactivated transducers and 1=activated transducers). For example, at to, C1=0, indicates that all the transducers of the circle C1 are deactivated at the instant t0, and at t3, C1=1, indicates that all the transducers of the circle C1 are activated at the instant t1.

It should be noted that the tables below are only used to illustrate sequences allowing a helix rotating about the transducer T0 to be acquired. These tables by no means limit the invention, other sequences can be contemplated by a person skilled in the art.

TABLE 7

| | t0 | t1 | t2 | t3 | t4 | t5 | t6 | t7 |
|---|---|---|---|---|---|---|---|---|
| T0 | 1 | 1 | 1 | 1 | 1 | 1 | 1 | 1 |
| B1 | 1 | 0 | 0 | 1 | 0 | 0 | 0 | 1 |
| B2 | 0 | 1 | 0 | 0 | 0 | 0 | 0 | 0 |
| B3 | 0 | 0 | 1 | 0 | 0 | 0 | 0 | 0 |
| B4 | 0 | 0 | 0 | 1 | 0 | 0 | 0 | 0 |
| B5 | 0 | 0 | 0 | 0 | 1 | 0 | 0 | 0 |
| B6 | 0 | 0 | 0 | 0 | 0 | 1 | 0 | 0 |
| B7 | 0 | 0 | 0 | 0 | 0 | 0 | 1 | 0 |
| B8 | 0 | 0 | 0 | 0 | 0 | 0 | 0 | 1 |

TABLE 8

| | t0 | t1 | t2 | t3 | t4 | t5 | t6 | t7 |
|---|---|---|---|---|---|---|---|---|
| T0 | 1 | 1 | 1 | 1 | 1 | 1 | 1 | 1 |
| B1 | 1 | 0 | 0 | 0 | 1 | 0 | 0 | 1 |
| B2 | 0 | 1 | 0 | 0 | 0 | 1 | 0 | 0 |
| B3 | 0 | 0 | 1 | 0 | 0 | 0 | 1 | 0 |
| B4 | 0 | 0 | 0 | 1 | 0 | 0 | 0 | 1 |
| B5 | 1 | 0 | 0 | 0 | 1 | 0 | 0 | 0 |
| B6 | 0 | 1 | 0 | 0 | 0 | 1 | 0 | 0 |
| B7 | 0 | 0 | 1 | 0 | 0 | 0 | 1 | 0 |
| B8 | 0 | 0 | 0 | 1 | 0 | 0 | 0 | 1 |

TABLE 9

| | t0 | t1 | t2 | t3 | t4 | t5 | t6 | t7 |
|---|---|---|---|---|---|---|---|---|
| T0 | 1 | 1 | 1 | 1 | 1 | 1 | 1 | 1 |
| B1 | 1 | 0 | 0 | 0 | 0 | 0 | 0 | 1 |
| B2 | 1 | 1 | 0 | 0 | 0 | 0 | 0 | 0 |
| B3 | 0 | 1 | 1 | 0 | 0 | 0 | 0 | 0 |
| B4 | 0 | 0 | 1 | 1 | 0 | 0 | 0 | 0 |
| B5 | 0 | 0 | 0 | 1 | 1 | 0 | 0 | 0 |
| B6 | 0 | 0 | 0 | 0 | 1 | 1 | 0 | 0 |
| B7 | 0 | 0 | 0 | 0 | 0 | 1 | 1 | 0 |
| B8 | 0 | 0 | 0 | 0 | 0 | 0 | 1 | 1 |

These sequences can be repeated several times, and/or as many times as necessary. The instants t0, t1, t2, . . . , t8 can last from 0.1 milliseconds to 5 seconds each, depending on the use made of the stimulator. For example, when the transducers are loudspeakers, the instants t0, t1, t2, . . . , t8 can be of the order of one second. Also, if the transducers are ultrasound transducers, the instants t0, t1, t2, . . . , t8 can be of the order of one millisecond. The instants t0, t1, t2, . . . , t8 can have the same duration or can have different durations. The instants t0, t1, t2, . . . , t8 are in succession so that there are always active transducers.

FIG. 3c illustrates an acoustic wave beam shape acquired with an array M of transducers according to the third arrangement. The first wave shot is that of the transducers located on the branches B1-B5, the second shot is that of the transducers of the branches B2-B6, the third shot is that of the transducers of the branches B3-B7, the fourth shot is that of the branches B4-B8, and so on. This succession of shots corresponds to [Table 8]. The beam is thus made up of a series of waves in the form of a rotating helix that converges toward the target zone Zi. The beam is helicoid shaped.

In this case again, the entire surface of the target zone Zi is reached by the beam, but in a progressive manner. The target zone Zi will first receive the acoustic energy from the transducers of the branches B1-B5, then successively from the transducers of the other branches, with each shot reaching a strip or a localized portion of said zone. The inventor has found that reaching the target zone Zi in this localized and progressive manner allowed fast and deep penetration of the acoustic waves into the body of the individual and allowed fast and homogeneous stimulation of said target zone, in the sense that the acoustic energy is better distributed in said zone and is not highly focused in a single focal point. Also, in the case whereby the transducers are ultrasound transducers, the formation of very hot spots at the focal point F is avoided. The acoustic energy of said waves thus acts with enhanced effectiveness in the target zone Zi, which is better stimulated, notably compared to the aforementioned stimulators of the prior art.

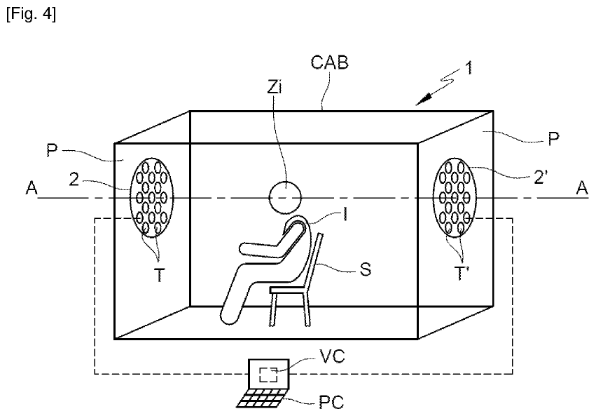

In FIGS. 3a, 3b and 3c, as in FIGS. 4, 5, 6a, 6b and 6c, the two emitters 2 and 2′ are symmetrically arranged in a mirror arrangement, and have the same acoustic axis A-A. This axis passes through the center of the matrices M, M′. The first array M of transducers T and the second array M′ of transducers T′ are identical. They face each other. The transducers T of the first array M diffuse a first beam Fo of acoustic waves and the transducers T′ of the second array M′ diffuse a second beam Fo′ of acoustic waves. The target zone Zi thus receives the acoustic energy of the two beams, so that it is further stimulated. In addition, it can be seen that the target zone Zi is stimulated from two distinct and opposite faces, thus increasing the effectiveness of the stimulator in order to stimulate said zone.

According to one embodiment, the unit UC activates the transducers T and T′ in accordance with the same sequence so that their beam Fo, Fo′ assumes the same shape. The acoustic waves emitted by the transducers T can have the same features as those emitted by the transducers T′. In this case, the target zone Zi is stimulated in the same way irrespective of its stimulated face, which can be advantageous when said zone has the same features (for example, in terms of density and/or shape) from one face to the next.

According to another embodiment, the unit UC activates the transducers T in accordance with a first sequence and the transducers T′ in accordance with a second sequence different from said first sequence, so that their beam Fo, Fo′ does not assume the same shape. For example, in the case of the first embodiment (FIG. 1a), the transducers T of the array M can be activated according to [Table 2] and those T′ of the array M′ can be activated according to [Table 1]. In the case of the second embodiment (FIG. 1b), the transducers T can be activated according to [Table 5] and the transducers T′ can be activated according to [Table 6]. In the case of the third embodiment (FIG. 1c), the transducers T can be activated according to [Table 8] and the transducers T′ can be activated according to [Table 7]. This notably can be advantageous when the target zone does not have the same features (for example, in terms of density and/or shape) from one face to the next and/or when the stimulation must be differentiated from one face to the next. For the same reasons, the matrices M and M′ can have different arrangements of transducers so that their beam Fo, Fo′ does not assume the same shape.

According to yet another embodiment, the acoustic waves emitted by the transducers T of the first array M have features distinct from those emitted by the transducers T′ of the second array M′ (whether or not their beam Fo, Fo′ assumes the same shape). For example, when the transducers are loudspeakers, the sound waves that they emit can have different frequencies and/or acoustic levels from one array to the next. Different sounds also can be emitted: for example, high-pitched sounds emitted from the first array M and bass sounds emitted from the second array M′ (or vice versa). According to another example, when ultrasound transducers are used, the ultrasound frequency of the waves of the first beam can be different from the ultrasound frequency of the waves of the second beam. This is particularly advantageous for differentiating the stimulation from one face to the next of the target zone Zi.

According to yet another embodiment, the unit UC activates the transducers T of the first array M and the transducers T′ of the second array M′ with a time lag. For example, the activation sequence of the transducers T′ can be launched after or before the activation sequence of the transducers T, with a delay or an advance of 0.1 milliseconds to 1 second. The transducers T and T′ can be activated in accordance with the same sequence or with distinct sequences. Similarly, the acoustic waves emitted by the transducers T can have the same features or features distinct from those transmitted by the transducers T′. This time lag notably allows the stimulation of the faces of the target zone Zi to be differentiated over time.

### Stimulator: First Embodiment—FIG. 4

In this case, the first emitter 2 and the second emitter 2′ are installed on the walls P of a cabin CAB. This cabin is, for example, made up of a set of panels assembled together. The walls of the cabin CAB can be soundproofed so as to allow optimal diffusion of the acoustic waves inside said cabin. The cabin CAB is preferably closed when the stimulator is used. By way of an example, the length of the cabin CAB ranges between 1 m and 5 m, its width ranges between 1 m and 5 m and its height ranges between 2 m and 3 m.

The emitters 2, 2′ are acoustic enclosures and the transducers T, T′ are loudspeakers (first embodiment), so that the emitted acoustic waves are sound waves. The enclosures 2 and 2′ are symmetrically arranged in a mirror arrangement, as in FIGS. 3a to 3c. According to one embodiment, the target zone Zi is located in the acoustic axis A-A of the two emitters 2, 2′ and midway from said emitters. In FIG. 4, the target zone Zi is the head of the individual I. However, the target zone can be another part of the body of the individual, for example, their torso, or even their whole body.

The cabin CAB is advantageously provided with a seat S, which can assume various forms, such as a stool, an armchair, a chair, etc. The seat S is placed in the cabin CAB so that, when the individual I is seated thereon, the first enclosure 2 is located opposite said individual and the second enclosure 2′ is located at the back of said individual. According to one embodiment, the seat S is positioned midway between the enclosures 2, 2′ and is height adjustable so as to be able to position the head of the individual I in the acoustic axis A-A. According to an alternative embodiment, the enclosures 2, 2′ are height adjustable so as to be able to position the acoustic axis A-A at the level of the head of the individual I. According to another embodiment, the enclosures 2, 2′ are installed in a room, fixed on walls or on tripods.

The management unit UC can be integrated in a computer PC (or a tablet, or a smartphone) connected in a wired or wireless manner (for example, by a Wi-Fi® or a Bluetooth® link) to the enclosures 2, 2′. An operator can thus easily control the operation of the enclosures 2, 2′ and of their transducers.

This stimulator 1 is particularly suitable for a non-therapeutic application, notably for a relaxation session of the individual. According to one embodiment, the individual is a healthy individual in the sense that they are not afflicted with a neurological disease. The audible sounds emitted by the emitters 2, 2′ can be music, songs, sounds of nature (noises of streams, birds, rain, waves, etc.), recordings of vibrations from bells, Tibetan gongs, tuning forks, etc. Due to the mirror arrangement of the emitters 2, 2′ and the specific diffusion of the sound waves, as described with reference to FIGS. 1a-1c and 3a-33c, the inventor has observed that the acoustic energy of said waves allowed fast access to a kind of physical and mental well-being. Good results in terms of relaxation and feelings for the user are acquired at the end of a session ranging from 30 minutes to 1 hour. In order to extend this effect of well-being, it is possible to contemplate a session of 10 sessions of 30 to 60 minutes each, with each session being spaced apart by 1 to 10 days. In order to assess the effect of the stimulator on the physical and mental well-being of the individual, the Warwick-Edinburgh mental well-being scale (WEMWBS) can be used. This WEMWBS scale was developed in 2007 by Warwick and Edinburgh. It includes 14 items on a Likert scale of 5 (1: never, 2: rarely, 3: sometimes, 4: often, 5: all the time). Its aim is to assess hedonistic (state of happiness and satisfaction with life) and eudemonistic (positive psychological functioning, satisfactory relationships with others, self-actualization and acceptance) well-being. The higher the score, the higher the psychological well-being of the individual. Individuals completed the WEMWBS scale two days before a stimulation session, then one day after. It has been found that the acquired score increased on average by 20% for a 30 minute session, and could increase by 50% after a series of 5 sessions lasting 30 minutes each, with each session being spaced apart by 2 days.

The inventor has also surprisingly found that this stimulator is also suitable for implementing a method for the therapeutic treatment of neurological diseases such as memory loss, Alzheimer's disease, Parkinson's disease, sleep disorders, by stimulating brain cells. It has been found that symptoms associated with these diseases could be significantly reduced when the individual was stimulated by the acoustic energy of this installation.

The following protocol was set up in order to test the effectiveness of the stimulator:
Type of sound emitted by the stimulator: classical music (Mozart, Bach, Handel and Chopin).
Duration of the sessions: 5 sessions of 15 minutes each, with each session being spaced apart by 24 hours.
Group studied: two groups A and B. Each group is made up of 10 patients 60 years old or older, with one half being diagnosed with Alzheimer's, and the other half being diagnosed with Parkinson's. The patients live at home and fall within GIR 4 and 3 (autonomy loss index computed from the AGGIR grid). All patients have continued to follow, for the 5 test days, the same therapeutic activities organized by workshops. Only patients from group B were also treated by the stimulator.
Effects observed following the treatment by the stimulator: reduction in anxiety and stress, improvement in language, increase in concentration, improvement in motor skills.
Assessment means used:
   Assessment of Anxiety and Stress: “State and Trait Anxiety Index” (STAI) tool. This tool is structured in two distinct scales in order to assess state anxiety and trait anxiety. They both include 20 items in the form of a Likert scale of 4 (with 1 indicating the lowest degree of anxiety and 4 indicating the highest degree of anxiety).
   Assessment of Language: GRBAS Scale. This scale is made up of six parameters: Grade (degrees of vocal abnormalities), Roughness (hoarseness, vibration irregularity), Breathiness (breath, presence of a glottic leakage), Asthenia (voice fatigue, hypophonia, hypokinesia), Strain (forced, hyperkinesia), Instability (variability of the voice quality over time or of one of the five preceding parameters). Each parameter is quantified by a Likert scale of 3 (0: normal, 1: light, 2: moderate, 3: severe).
   Assessment of concentration: automatic writing test (surname, forename, date, signature, Arabic numerals from 1 to 20, days and month) and copy test (having the patient copy a text).
   Motor skills assessment: SPPB (Short Physical Performance Battery) test. The result is the sum of the scores based on three criteria: balance test, walking speed test and chair sit-to-stand test. This test allows the physical performance of an individual to be assessed. Adding the scores of all the tests allows an overall performance score to be acquired.
      The patients from the two groups were assessed before the first session (TO) and on completion of the last session (T1).

The table below shows the results that were acquired. The average evolutions (as a percentage %) of the results (scores) of the assessments at T1 are shown.

TABLE 10

| | Group A | | Group B | |
|---|---|---|---|---|
| | (not treated by a stimulator) | | (treated by a stimulator) | |
| | T0 | T1 | T0 | T1 |
| Anxiety and | 0 | 10% reduction of | 0 | 40% reduction |
| stress | | the average of the | | of the average |
| (STAI tool) | | scores | | of the scores |
| Language | 0 | 5% reduction of the | 0 | 15% reduction |
| (GRBAS scale) | | average of the | | of the average |
| | | scores | | of the scores |
| Concentration | 0 | 0 | 1 | Significant |
| (Writing tests) | | | | observed |
| | | | | improvements |
| | | | | in 60% of |
| | | | | patients |
| Motor skills: | 0 | 0 | 1 | 20% increase |
| (Test SPPB) | | | | in the average |
| | | | | of the scores |

This stimulator 1 also can be simply used to listen to music or as an acoustic element of a home cinema. The individual would then benefit from good sound comfort and a unique listening experience, with the enclosures 2′, 2′ promoting a homogeneous sound ambience in the room and/or the cabin.

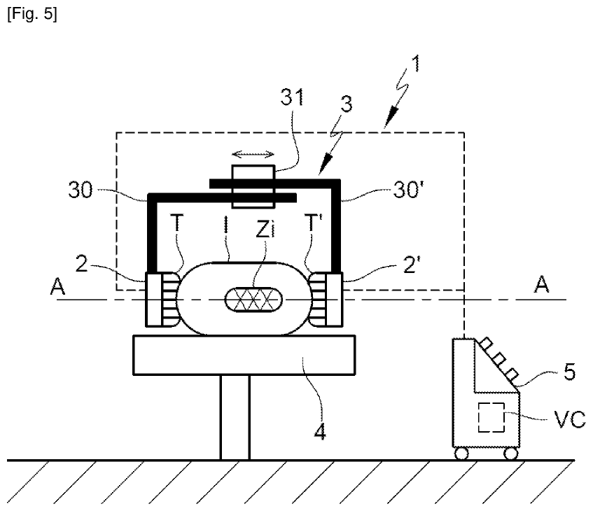

### Stimulator: Second Embodiment—FIG. 5

The transducers T, T′ in this case are therapeutic transducers (second embodiment). The emitters 2 and 2′ are therapeutic probes symmetrically arranged in a mirror arrangement, as in FIGS. 3a to 3c. According to one embodiment, the target zone Zi is located in the acoustic axis A-A of the two emitters 2, 2′. In FIG. 5, the target zone Zi is located inside the body of the individual I and is made up of, for example, a zone of diseased tissues such as a tumor or cells to be destroyed.

According to one embodiment, the first emitter 2 and the second emitter 2′ are fixed on the arms 30, 30′ of a support 3. The support 3 comprises a means 31 for adjusting the spacing between the arms 30, 30′ and therefore the spacing between the emitters 2, 2′. This means 31 is, for example, in the form of a rack. The adjustment of the spacing between the emitters 2, 2′ makes it possible to adapt to the morphology of the individual I and/or to the part of the body against which they are applied. The individual I is positioned on a platform 4, which can be, for example, in the form of a bed they are lying on. The management unit UC can be integrated into an electronic bay or carriage connected to the emitters 2, 2′. Means other than the arms 30, 30′ can be provided to adjust the spacing between the emitters 2, 2′, for example, a manual adjustment of each emitter being held in a hand of an operator. According to another embodiment, no adjustment means is provided, with the spacing between the emitters 2, 2′ being fixed.

Due to the mirror arrangement of the emitters 2, 2′ and the specific diffusion of the waves as described with reference to FIGS. 1a-1c and 3a-33c, the target zone Z1 receives the acoustic energy of the two beams Fo and Fo′, from two distinct and opposite faces, which allows said zone to be effectively treated. More specifically, the waves cause the localized and remote elevation of the temperature of the target zone Zi for necrosis of the diseased tissues without touching the surrounding tissues. Furthermore, being able to treat the target zone Zi from two distinct and opposite faces allows a larger volume of diseased tissues to be treated. Furthermore, the increase in temperature of the target zone Zi is faster, which allows the treatment to be accelerated.

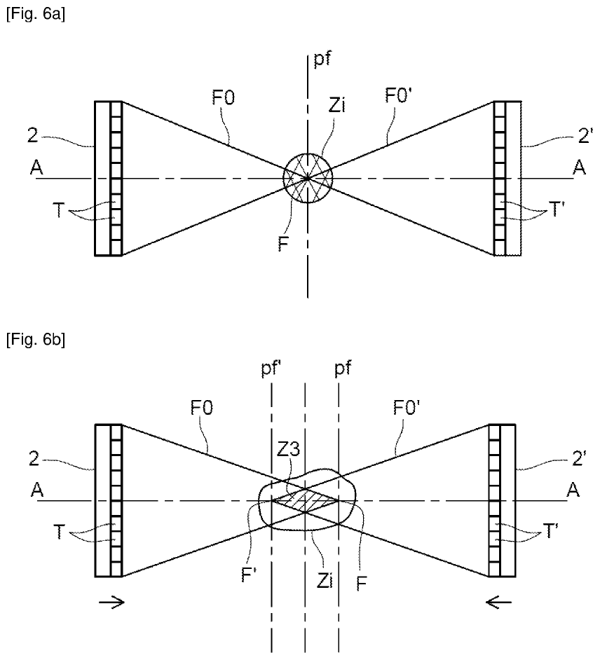

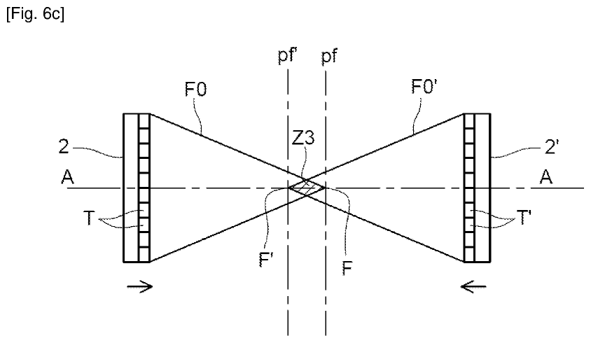

In FIG. 6a, the first beam Fo of acoustic waves emitted by the first emitter 2 and the second beam Fo′ of acoustic waves emitted by the second emitter 2′ are advantageously focused so that they converge toward the same focal point F located on the acoustic axis A-A of the emitters 2, 2′, at an equal distance from said emitters, in the same focal plane Pf. The concentration of the acoustic energy is then maximal at the focal point F, which allows the target zone Zi to be very quickly stimulated at this level.

However, it is possible to adjust the focal length of the emitters 2, 2′ so that they do not have the same focal point. In FIG. 6b, the first beam Fo converges toward a first focal point F and the second beam Fo′ converges toward a second focal point F′, which is distinct from said first focal point. The two focal points F and F′ are located on the acoustic axis A-A of the emitters 2, 2′.

The first beam F makes it possible to act in a first focal zone located in the focal plane Pf of the first focal point F and the second beam F′ makes it possible to act in a second distinct focal zone located in the focal plane Pf′ of the second focal point F′. A third focal zone Z3 for stimulation corresponds to the overlapping zone where the first beam Fo and the second beam Fo′ intersect.

Several zones thus can be stimulated, and notably:
a first focal zone located at the first focal point F;
a second focal zone located at the second focal point F′;
a third focal zone Z3 located between the first focal point F and the second focal point F′, where the first beam Fo and the second beam Fo′ overlap.

In the third zone Z3, the acoustic energies of the two beams Fo and Fo′ combine to effectively stimulate said zone. This stimulation is also particularly homogeneous and fast due to the specific diffusion of the acoustic waves. It is therefore possible to treat a larger target zone Zi (and notably a larger amount of lesions in the case of therapeutic transducers): in the first focal zone, in the second focal zone and in the third focal zone.

The position and/or the dimensions of the third zone Z3 can be adjusted mechanically by modifying the spacing between the emitters 2, 2′. It is thus possible to differentiate the focal points F and F′, 2′.

The position and/or the dimensions of the third zone Z3 also can be adjusted in another way, notably electronically. For example, when the transducers T, T′ are loudspeakers integrating HSS® technology, the ultrasound carrier frequency can be modulated to move the focal point F and/or F′. Also, in the case where the transducers T, T′ are therapeutic transducers, the convergent acoustic lenses of said transducers can be modified to adapt the position of the focal point F and/or F′. With this type of solution, it is possible to off-center the position of the third zone Z3. In FIG. 6c, the first focal point F is in the middle of the acoustic axis A-A (in the plane of symmetry of the stimulator and/or equidistant from the emitters 2, 2′), while the second focal point F′ is located beyond. As a result, the third zone Z3 is off-centered from this midpoint.

The use of emitters 2, 2′ with an adjustable focal length therefore allows the acoustic energies to be concentrated in different specifically selected target zones of the zone of interest Zi.

According to an embodiment not covered by the present invention, the stimulator comprises a single acoustic energy emitter as described according to one of the preceding embodiments.

The arrangement of the various elements and/or means and/or steps of the invention, in the embodiments described above, should not be understood as requiring such an arrangement in all implementations. Other alternative embodiments can be provided. Notably, the therapeutic transducers can be infrasound transducers.

Furthermore, one or more features only disclosed in one embodiment can be combined with one or more other features only disclosed in another embodiment. Similarly, one or more features only disclosed in one embodiment can be generalized to the other embodiments, even if this or these features are described only in combination with other features.

1-15. (canceled)

16. A stimulator configured to apply acoustic energy to a target zone of an individual, comprising:
a first acoustic energy emitter equipped with a first array of electroacoustic transducers and a second acoustic energy emitter equipped with a second array of electroacoustic transducers, said emitters symmetrically arranged in a mirror arrangement, with the first array facing the second array;
an electronic management unit configured to activate the transducers of the first array so that said transducers diffuse a first beam of acoustic waves toward a target zone of the individual, and to activate the transducers of the second array so that said transducers diffuse a second beam of acoustic waves toward said target zone; and
the activation of the transducers of the first array and of the second array is sequenced so that said transducers of each emitter are activated in the form of successive concentric circles or rings with different diameters or in the form of a spiral or in the form of a helix rotating about an acoustic axis of the two emitters.

17. The stimulator as claimed in claim 16, wherein the acoustic waves are sound waves.

18. The stimulator as claimed in claim 16, wherein the first emitter and the second emitter are installed on the walls of a cabin.

19. The stimulator as claimed in claim 18, wherein a seat is placed in the cabin so that, when the individual is seated on said seat, the first emitter is located opposite said individual and the second emitter is located at the back of said individual.

20. The stimulator as claimed in claim 16, wherein the acoustic waves are ultrasound waves.

21. The stimulator as claimed in claim 20, wherein the ultrasound frequency of the waves of the first beam is different from the ultrasound frequency of the waves of the second beam.

22. The stimulator as claimed in claim 16, wherein the acoustic waves emitted by the transducers of the first array have the same features as the acoustic waves emitted by the transducers of the second array.

23. The stimulator as claimed in claim 16, wherein the transducers of the first array and the transducers of the second array are directive diffusion electroacoustic transducers.

24. The stimulator as claimed in claim 23, wherein:
the first beam converges toward a first focal point to receive a stimulation in a first focal zone;
the second beam converges toward a second focal point, which is distinct from said first focal point, to receive a stimulation in a second focal zone; and
a third stimulation focal zone corresponds to an overlapping zone where the first beam and the second beam intersect.

25. The stimulator as claimed in claim 16, wherein the target zone is located in the acoustic axis of the two emitters and midway from said emitters.

26. The stimulator as claimed in claim 16, wherein the electronic management unit is configured to activate the transducers of the first array and the transducers of the second array in accordance with the same sequence.

27. The stimulator as claimed in claim 16, wherein the electronic management unit is configured to activate the transducers of the first array in accordance with a first sequence and the transducers of the second array in accordance with a second sequence different from said first sequence.

28. The stimulator as claimed in claim 16, wherein the electronic management unit is configured to activate the transducers of the first array and the transducers of the second array with a time lag.

29. A non-therapeutic method for applying acoustic energy to a target zone of an individual, the method comprising:
equipping a first acoustic energy emitter with a first array of electroacoustic transducers and a second acoustic energy emitter with a second array of electroacoustic transducers;
symmetrically positioning the first emitter and the second emitter in a mirror arrangement, with the first array (M) facing the second array;
activating the transducers of the first array so that said transducers diffuse a first acoustic wave beam toward a target zone of the individual, and activating the transducers of the second array so that said transducers diffuse a second acoustic wave beam toward said target zone, which acoustic waves are sound waves; and
sequencing the activation of the transducers of the first array and of the second array so that said transducers of each emitter are activated in the form of successive concentric circles, disks, or rings with different diameters, or in the form of a spiral, or in the form of a helix rotating about an acoustic axis of the two emitters.

30. A device for the therapeutic treatment of a neurological disease comprising a brain cell stimulator, wherein said brain cell stimulator is the stimulator of claim 16.
